(19) United States
(12) Patent Application Publication
(10) Pub. No.: US 2013/0245445 A1
(43) Pub. Date: Sep. 19, 2013

(54) ULTRASONIC DIAGNOSTIC APPARATUS AND METHOD OF CONTROLLING THE SAME

(71) Applicants: KABUSHIKI KAISHA TOSHIBA, TOKYO (JP); TOSHIBA MEDICAL SYSTEMS CORPORATION, OTAWARA-SHI (JP)

(72) Inventors: Akihiro KAKEE, Nasushiobara-shi (JP); Kuramitsu NISHIHARA, Otawara-shi (JP)

(73) Assignees: TOSHIBA MEDICAL SYSTEMS CORPORATION, OTAWARA-SHI (JP); KABUSHIKI KAISHA TOSHIBA, TOKYO (JP)

(21) Appl. No.: 13/833,126

(22) Filed: Mar. 15, 2013

(30) Foreign Application Priority Data
Mar. 15, 2012 (JP) 2012058842
Mar. 12, 2013 (JP) 2013-048656

(51) Int. Cl. A61B 8/00

(52) U.S. Cl. CPC A61B 8/54
USPC 600/440; 600/443

(57) ABSTRACT

According to one embodiment, an ultrasonic diagnostic apparatus includes a phase difference detection unit, a correction unit and an addition unit. The phase difference detection unit detects a phase difference between temporally adjacent reception signals of a plurality of reception signals on each scanning line at each sampling point on each scanning line. The correction unit performs correction processing including at least time delay correction for at least one of a plurality of reception signals for each scanning line based on the phase difference at each sampling point on each scanning line. The addition unit adds, for each scanning line, the plurality of reception signals including a reception signal subjected to the correction processing.

### CROSS-REFERENCE TO RELATED APPLICATIONS

This application is based upon and claims the benefit of priority from Japanese Patent Applications No. 2012-058842, filed Mar. 15, 2012; and No. 2013-048656, filed Mar. 12, 2013, the entire contents of all of which are incorporated herein by reference.

### FIELD

Embodiments described herein relate generally to an ultrasonic diagnostic apparatus and a method of controlling the same.

### BACKGROUND

An ultrasonic diagnostic apparatus is an apparatus which transmits ultrasonic beams to a living body, receives reflected waves, and generates an image of the in vivo tissue (diagnosis target region) by applying the principle of the pulse reflection method to the received reflected waves. The ultrasonic diagnostic apparatus has features such as noninvasiveness, compactness, and real-time display, and hence is widely used in medical fields.

Such an ultrasonic diagnostic apparatus includes, for example, a transmission/reception unit, a combining unit, an image generation unit, and a control unit. When using a polarity reversal technique, the transmission/reception unit performs one or more sets of ultrasonic transmission/reception (transmission of an ultrasonic beam and reception of reflected wave data), each including two times of ultrasonic transmission/reception, while reversing the phase polarity on the same scanning line (i.e., in the same transmission/reception direction). The combining unit combines reception signals as reflected wave data received as a result of a plurality of times of ultrasonic transmission/reception in each set. In the following description, a series of processing of executing a plurality of times of ultrasonic transmission/reception on the same scanning line and combining acquired reception signals in this manner will be referred to as a “combining scan” hereinafter.

In general, in examination by the ultrasonic diagnostic apparatus, the sensitivity in a deep portion tends to be low. As a measure for improving the sensitivity in a deep portion, it is conceivable to, for example, decrease the frequency of an ultrasonic pulse. In this case, however, the spatial resolution decreases. In addition, it is conceivable to use the normal mode of imaging fundamental waves while inhibiting the use of the mode of imaging harmonic components (this mode will be referred to as a harmonic imaging mode hereinafter). In this case, however, it is not possible to obtain the artifact reducing effect which can be obtained by the harmonic imaging mode.

As disclosed in, for example, Jpn. Pat. Appln. KOKAI Publication No. 2008-178470, there has been provided a technique of improving the sensitivity in a deep portion by improving the SNR (Signal to Noise Ratio) by increasing the number of data used per scanning line.

Increasing the number of data used per scanning line will make combining scan processing susceptible to the influences of the respiration and pulsation of an object. This can lead to a degradation in spatial resolution due to motion artifacts and “blurring”.

Note that there is provided a technique of detecting the motion amount of a diagnosis target region (to be simply abbreviated as a “target region” hereinafter) and intermittently performing a combining scan only in a time zone in which the amount of motion of the target region is small, based on the detection result. This technique, however, can be used only in a time zone in which the influences of respiration and pulsation are small.

In consideration of the above situation, there are provided an ultrasonic diagnostic apparatus which implements an improvement in SNR and an increase in spatial resolution, and a method of controlling the apparatus.

### BRIEF DESCRIPTION OF THE DRAWINGS

FIG. 1 is a block diagram showing an example of the arrangement of an ultrasonic diagnostic apparatus according to an embodiment;

FIG. 2 is a block diagram showing an example of the arrangement of a B-mode processing unit;

FIG. 3 is block diagram showing an example of the arrangement of a Doppler processing unit;

FIG. 4 is a graph showing the reception spectrum intensities of the fundamental wave components and harmonic components of reception signals;

FIG. 5 is a flowchart for ultrasonic image generation processing by the ultrasonic diagnostic apparatus according to an embodiment;

FIG. 6 is a view showing the ultrasonic image generated by a conventional ultrasonic diagnostic apparatus; and

FIG. 7 is a view showing the ultrasonic image generated by the ultrasonic diagnostic apparatus according to the embodiment.

### DETAILED DESCRIPTION

In general, according to one embodiment, an ultrasonic diagnostic apparatus includes a transmission/reception unit, a phase difference detection unit, a correction unit, an addition unit, and an image generation unit. The transmission/reception unit acquires, for each scanning line, a plurality of reception signals corresponding to a plurality of ultrasonic beams by performing a plurality of times of ultrasonic transmission/reception on the same scanning line. The phase difference detection unit detects a phase difference between temporally adjacent reception signals of a plurality of reception signals on each scanning line at each sampling point on each scanning line. The correction unit performs correction processing including at least time delay correction for at least one of a plurality of reception signals for each scanning line based on the phase difference at each sampling point on each scanning line. The addition unit adds, for each scanning line, the plurality of reception signals including a reception signal subjected to the correction processing. The image generation unit generates a first image from the reception signals added for each scanning line.

An ultrasonic diagnostic apparatus according to an embodiment will be described below.

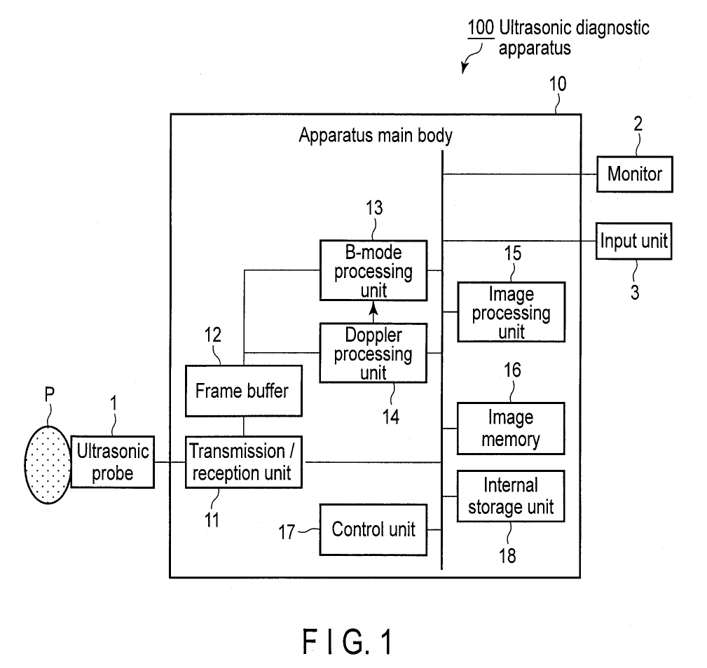

FIG. 1 is a block diagram showing an example of the arrangement of the ultrasonic diagnostic apparatus according to this embodiment. As shown in FIG. 1, an ultrasonic diagnostic apparatus 100 includes an ultrasonic probe 1, a monitor 2, an input unit 3, and an apparatus main body 10.

The ultrasonic probe 1 includes a plurality of piezoelectric transducers. These ultrasonic transducers generate ultrasonic pulses based on the driving signals supplied from a transmission/reception unit 11 of the apparatus main body 10 (to be described later). The ultrasonic transducers also receive reflected waves from an object P and convert the waves into electrical signals. The ultrasonic probe 1 includes matching layers provided for the piezoelectric transducers and a backing member which prevents ultrasonic waves from propagating backward from the piezoelectric transducers.

When the ultrasonic probe 1 transmits ultrasonic waves to the object P, the ultrasonic pulses are sequentially reflected by a discontinuity surface of acoustic impedance of the in vivo tissue of the object. The plurality of piezoelectric transducers of the ultrasonic probe 1 receive the ultrasonic waves as echo signals. The amplitude of each echo signal depends on an acoustic impedance difference on the discontinuity surface by which the ultrasonic pulse is reflected. The echo signal produced when a transmitted ultrasonic pulse is reflected by the surface of a moving blood flow, cardiac wall, or the like is subjected to a frequency shift depending on the velocity component of the moving body in the ultrasonic transmission direction due to the Doppler effect.

The monitor 2 displays a GUI (Graphical User Interface) on which the operator of the ultrasonic diagnostic apparatus 100 inputs various types of instructions and setting requests with the input unit 3, the ultrasonic images generated by the apparatus main body 10, analysis results, and the like.

The input unit 3 includes a mouse, keyboard, buttons, panel switches, touch command screen, foot switch, and trackball, and is connected to the apparatus main body 10. The input unit 3 accepts various types of instructions and setting requests from the operator of the ultrasonic diagnostic apparatus 100, and transfers the accepted instructions and setting requests to the apparatus main body 10.

The apparatus main body 10 generates an ultrasonic image based on the reflected waves received by the ultrasonic probe 1. As shown in FIG. 1, the apparatus main body 10 includes the transmission/reception unit 11, a frame buffer 12, a B-mode processing unit 13, a Doppler processing unit 14, an image processing unit 15, an image memory 16, a control unit 17, and an internal storage unit 18.

The transmission/reception unit 11 includes a trigger generation circuit, a transmission delay circuit, and a pulser circuit, and supplies driving signals to the ultrasonic probe 1. The pulser circuit repeatedly generates rate pulses for the formation of ultrasonic waves at a predetermined PRF (Pulse Repetition Frequency). Note that a PRF is also called a rate frequency. The transmission delay circuit gives each rate pulse generated by the pulser circuit a transmission delay time, for each piezoelectric transducer, necessary to focus the ultrasonic pulses generated from the ultrasonic probe 1 into a beam and determine transmission directivity. The trigger generation circuit applies a driving signal (driving pulse) to the ultrasonic probe 1 at the timing based on a rate pulse. That is, the transmission delay circuit arbitrarily adjusts the transmission direction from the piezoelectric transducer surface by changing the transmission delay time given to each rate pulse.

The transmission/reception unit 11 has a function of capable of instantaneously changing a transmission frequency, a transmission driving voltage, and the like to execute a predetermined sequence based on instructions from the control unit 17 (to be described later). In particular, a transmission driving voltage can be changed by a linear amplifier type transmission circuit capable of instantaneously switching its values or a mechanism of electrically switching a plurality of power supply units.

The transmission/reception unit 11 includes an amplifier circuit, A/D (Analog/Digital) converter, reception delay circuit, and a quadrature detection circuit, and generates a reception signal by performing various types of processing for the reception signal (reflected wave data) received by the ultrasonic probe 1.

The amplifier circuit performs gain correction processing by amplifying a reception signal for each channel.

The A/D converter A/D-converts the reception signal having undergone gain correction processing. The reception delay circuit gives digital data a reception delay time necessary to determine reception directivity.

The quadrature detection circuit converts a reception signal into an I (In-phase) signal and a Q (Quadrature-phase) signal in the baseband. The quadrature detection circuit stores the I and Q signals (to be collectively referred to as IQ signals hereinafter) as a reception signal to the frame buffer 12 on the subsequent stage.

Note that this quadrature detection circuit is not an essential constituent element. If no quadrature detection circuit is provided, an RF (Radio Frequency) signal as a reception signal before processing by the quadrature detection circuit is stored as a reception signal in the frame buffer 12 on the subsequent stage. Delay correction processing (to be described later) can also be applied to RF signals. Although the following will exemplify the use of I and Q signals as reception signals, the application of this embodiment to RF signals will be described as needed.

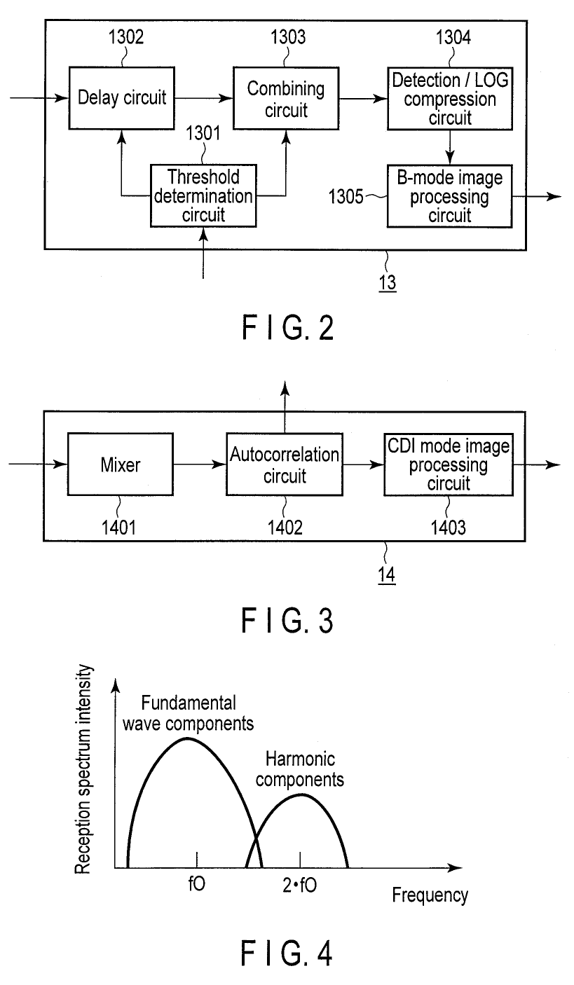

The B-mode processing unit 13 generates data (B-mode data) whose signal intensity is expressed by a luminance level from the reception signal output from the transmission/reception unit 11. FIG. 2 is a block diagram showing an example of the arrangement of the B-mode processing unit 13. As shown in FIG. 2, the B-mode processing unit 13 includes a threshold determination circuit 1301, a delay circuit 1302, a combining circuit 1303, a detection/LOG compression circuit 1304, and a B-mode image processing circuit 1305.

The threshold determination circuit 1301 compares phase difference information Δθ output from the Doppler processing unit 14 (to be described later) with a “predetermined threshold” stored in, for example, a memory (not shown) of the threshold determination circuit 1301, and determines for each ultrasonic beam, based on the comparison result, whether to perform delay correction processing by the delay circuit 1302.

More specifically, the threshold determination circuit 1301 determines whether, for example, the value indicated by the phase difference information Δθ according to each ultrasonic beam is equal to or less than “the upper limit value (first threshold) of allowable phase differences”, generates a control signal based on the determination result, and outputs the signal to the combining circuit 1303.

This control signal is a signal for controlling the combining circuit 1303 so as not to use, for combining processing (addition processing), any data inadequate as data to be used for the combining processing (to be referred to as inadequate data hereinafter) because the data has a large phase shift even if delay correction processing is applied to it.

The delay circuit 1302 calculates a delay amount for each ultrasonic beam with reference to a specific (e.g., the first) ultrasonic beam based on the phase difference information Δθ output from an autocorrelation circuit 1402, and corrects the phase of each reception signal by applying delay correction processing (phase delay correction/time delay correction) to the signal by the delay amount. This correction will eliminate the “phase shifts between the reception signals” due to, for example, the respiration, pulsation, and the like of a patient.

When omitting the quadrature detection circuit and using an RF signal as a reception signal, the apparatus executes delay correction processing targeted at only time delay correction.

More specifically, a delay amount for the nth rate of a plurality of times of ultrasonic transmission/reception on a given scanning line is calculated as the integral value of the phase differences between the first rate and the second to (n−1)th rates or calculated by multiplying average phase×(n−1) by the period of a reception center frequency. Note that when performing autocorrelation processing, the apparatus may calculate a delay amount for each sampling point (or each pixel) on each scanning line upon spatially thinning out data and interpolate each delay amount from actually measured delay amounts at nearby points. In addition, a phase difference to be used is not limited to an average phase difference and may be a representative phase difference, median phase difference, or the like.

Since it is not necessary to perform delay correction processing for inadequate data which is not used for combining processing, the threshold determination circuit 1301 may generate a control signal for controlling the delay circuit 1302 to inhibit it from applying delay correction processing to inadequate data, and output the signal to the delay circuit 1302.

The combining circuit 1303 combines (adds), for each scanning line, the reception signal phase-corrected by the delay circuit 1302 and reception signals which have not been excluded as inadequate data. That is, the combining circuit 1303 performs processing in accordance with the control signal output from the threshold determination circuit 1301, and hence does not use the above inadequate data for combining processing. As a consequence, the combining circuit 1303 obtains an addition signal for each scanning line by adding reception signals according to a plurality of times of ultrasonic transmission/reception on each scanning line.

The detection/LOG compression circuit 1304 receives a reception signal from the combining circuit 1303 and performs detection processing and LOG compression processing. When omitting the quadrature detection circuit and using an RF signal as a reception signal, the apparatus causes the detection/LOG compression circuit 1304 to execute envelope detection.

The B-mode image processing circuit 1305 receives a reception signal from the detection/LOG compression circuit 1304 and generates data (B-mode data) whose signal intensity is expressed by a luminance level.

The Doppler processing unit 14 includes a mixer 1401, the autocorrelation circuit 1402, and a CDI (Color Doppler Imaging) mode image processing circuit 1403, as shown in FIG. 3. FIG. 3 is a block diagram showing an example of the arrangement of the Doppler processing unit 14.

The mixer 1401 changes the reception center frequency with respect to the reception signals received from the transmission/reception unit 11. The processing performed by the mixer 1401 is the processing for improving the accuracy of “motion (velocity) detection processing of a biometric signal using the Doppler effect” by the CDI mode image processing circuit 1403.

In other words, the mixer 1401 is a circuit for shifting the baseband of a reception signal to supply fundamental wave components to CDI processing by the CDI mode image processing circuit 1403.

FIG. 4 is a graph showing the reception spectrum intensities of the fundamental wave components and harmonic components of reception signals. As shown in FIG. 4, considering the spectrum distribution of high-frequency components, i.e., harmonic components, it is obvious that they are greatly influenced by attenuation at the time of propagation. In this embodiment, in consideration of such a situation, the mixer 1401 sets a reception center frequency so as to use fundamental wave components for CDI processing, thereby broadening the detectable velocity range.

The processing by the mixer 1401 improves the “motion detection” performance of the CDI mode image processing circuit 1403, but is not essential. It is therefore possible to omit the mixer 1401. In this case, the CDI mode image processing circuit 1403 performs processing by using the frequencies of harmonic signals.

The autocorrelation circuit 1402 performs known autocorrelation processing to detect the phase difference between the temporally adjacent reception signals of ultrasonic beams among the reception signals of ultrasonic beams associated with a plurality of times of ultrasonic transmission/reception on the same scanning line. The autocorrelation circuit 1402 generates the phase difference information Δθ representing the detected phase difference. In addition, the autocorrelation circuit 1402 calculates time difference information Δt between the temporally adjacent reception signals from the phase difference information Δθ between the temporally adjacent reception signals and the center frequency of the reception signals.

In other words, the autocorrelation circuit 1402 detects the phase difference between adjacent ultrasonic beams among reception signals at the respective sampling points or pixels (i.e., at the respective depths of the respective ultrasonic beams) on each scanning line, and calculates the phase difference information Δθ and the time difference information Δt. The phase difference information Δθ and time difference information Δt generated in this manner are output to the B-mode processing unit 13.

The CDI mode image processing circuit 1403 performs CDI using the Doppler effect to frequency-analyze velocity information from the reception signal received from the transmission/reception unit 11, extract a blood flow or tissue owing to the Doppler effect and a contrast medium echo component, and extract moving object information such as mean velocities, variances, powers, and the like at multiple points, thereby generating data (Doppler data).

The image processing unit 15 generates an ultrasonic image from the B-mode data generated by the B-mode processing unit 13 and the Doppler data generated by the Doppler processing unit 14. More specifically, the image processing unit 15 generates a B-mode image from B-mode data, and generates a Doppler image from Doppler data.

The image processing unit 15 performs conversion (scan conversion) of a scanning line signal string for ultrasonic scanning into a scanning line signal string in a general video format typified by a TV format, and generates an ultrasonic image (a B-mode image, a Doppler image, or an image obtained by superimposing a B-mode image and a Doppler image) as an ultrasonic image.

The image memory 16 is a memory for storing the ultrasonic images generated by the image processing unit 15 and the images generated by processing ultrasonic images. For example, after diagnosis, the operator can call up images recorded during examination, and can play back them as still images or as a moving image using a plurality of images. The image memory 16 also stores image luminance signals after passing through the transmission/reception unit 11, other raw data, image data acquired via a network, and the like, as needed.

The control unit 17 controls the overall processing in the ultrasonic diagnostic apparatus 100. More specifically, the control unit 17 controls the processing performed by the transmission/reception unit 11, the B-mode processing unit 13, the Doppler processing unit 14, and the image processing unit 15 and performs control to display ultrasonic images stored in the image memory 16 and the like on the monitor 2 based on various types of instructions and setting requests input by the operator via the input unit 3 and various types of programs and various types of setting information read from the internal storage unit 18.

The internal storage unit 18 stores apparatus control programs for performing ultrasonic transmission/reception, image processing, and display processing, diagnosis information (e.g., patient IDs and findings by doctors), a diagnostic protocol, various types of data such as various types of setting information, and the like. The internal storage unit 18 is also used to archive images stored in the image memory 16, as needed.

The transmission/reception unit 11 and the like incorporated in the apparatus main body 10 are sometimes implemented by hardware such as integrated circuits and other times by software programs in the form of software modules.

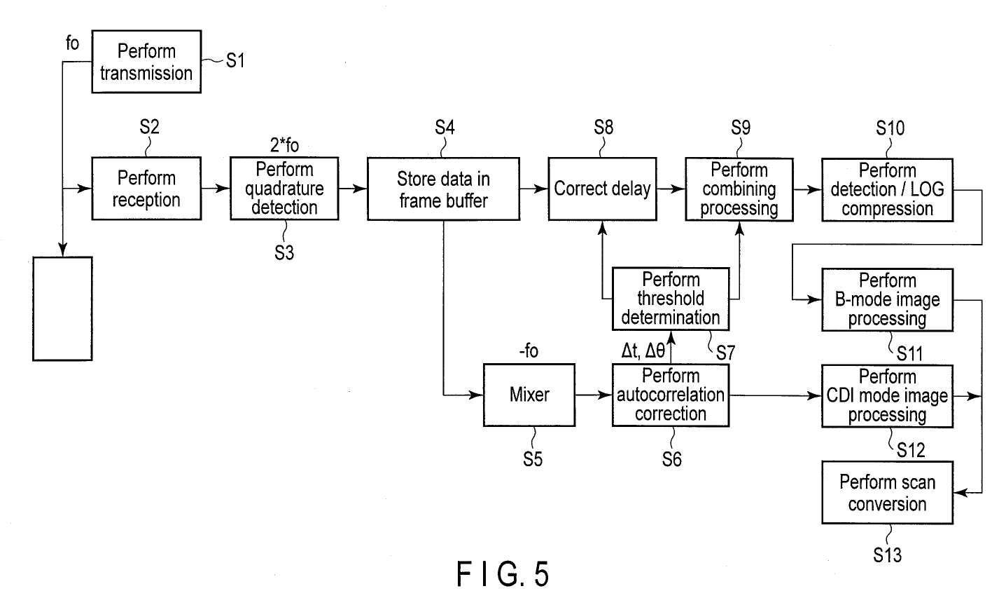

A procedure for ultrasonic image generation processing by the ultrasonic diagnostic apparatus 100 according to this embodiment will be described below. FIG. 5 is a flowchart for ultrasonic image generation processing by the ultrasonic diagnostic apparatus according to the embodiment. The following will exemplify the apparatus operating in the harmonic imaging mode of imaging harmonic components. The following will also exemplify the use of a technique of canceling fundamental wave components by reversing the phase polarity of an ultrasonic beam (to be referred to as a polarity reversal technique hereinafter).

The principle of the polarity reversal technique will be described first. The polarity reversal technique is a technique of canceling fundamental wave components included in a reception signal and extracting harmonic components by performing ultrasonic transmission/reception at least twice on the same scanning line.

For example, the phase polarity of an ultrasonic beam is made positive in the first transmission, and the phase polarity in the first transmission is reversed in the second transmission. Adding reception signals obtained by two times of transmission/reception will cancel fundamental wave components because of opposite phases, but will make harmonic components generated during ultrasonic propagation enhance each other because they are in phase with each other.

In this case, the ultrasonic diagnostic apparatus 100 according to the first embodiment performs a plurality of sets of ultrasonic transmission/reception on the same scanning line, each including two times of ultrasonic transmission/reception, while reversing the phase polarity on the same scanning line. That is, one set of ultrasonic transmission/reception includes ultrasonic transmission/reception performed with the positive polarity and ultrasonic transmission/reception performed with the negative polarity. For example, the transmission/reception unit 11 performs four sets of ultrasonic transmission/reception, each including two times of ultrasonic transmission/reception.

When the ultrasonic diagnostic apparatus 100 starts examination, the control unit 17 reads initially set scan conditions from the internal storage unit 18, and starts a scan in accordance with the read initial settings. First of all, the transmission/reception unit 11 starts transmitting an ultrasonic beam in the normal mode (step S1) and receiving a reception signal as reflected wave data (step S2).

In this case, scan conditions are changed in accordance with situations at different times, e.g., regions to be scanned. Scan conditions include, for example, an ultrasonic transmission/reception mode, a PRF (Pulse Repetition Frequency), and a depth.

Subsequently, the transmission/reception unit 11 performs the respective types of processing described above for the reception signal received by the ultrasonic probe 1, and then causes the quadrature detection circuit to convert the signal into I and Q signals (to be collectively referred to as reception signals hereinafter) in the baseband (step S3). The frame buffer 12 then stores these signals (step S4). The mixer 1401 changes the reception center frequency setting into the center frequency of the fundamental wave components concerning the I and Q signals (step S5).

The autocorrelation circuit 1402 generates the phase difference information Δθ and the time difference information Δt by known autocorrelation processing based on reception signals (step S6), and outputs the information to the threshold determination circuit 1301. Note that when using the polarity reversal technique as in this case, the apparatus extracts reception signals according to only odd-numbered rates or even-numbered rates of a plurality of times of ultrasonic transmission/reception and performs autocorrelation processing between the rates.

Upon receiving the phase difference information Δθ and the time difference information Δt, the threshold determination circuit 1301 compares each information with a corresponding threshold, and determines, based on the comparison results, whether to perform delay correction (to be described in detail later) for each reception signal by using the delay circuit 1302 (step S7). The threshold determination circuit 1301 outputs the control signal generated based on the determination result to the combining circuit 1303.

On the other hand, the delay circuit 1302 reads out a reception signal from the frame buffer 12, calculates a delay amount from the phase difference information Δθ for each ultrasonic beam, and performs delay correction processing for each reception signal based on the delay amount (step S8), thereby eliminating “the phase shifts between the reception signals” due to the respiration, pulsation, and the like of the patient. The combining circuit 1303 performs combining processing for the reception signals subjected to correction of phase shifts due to the motion of a diagnosis target region in this manner (step S9).

The detection/LOG compression circuit 1304 then performs detection processing and LOG compression processing for this combined reception signal (step S10). The B-mode image processing circuit 1305 generates B-mode data (step S11).

In parallel with the above processing in steps S7 to S11 by the B-mode processing unit 13, the Doppler processing unit 14 generates Doppler data by using the CDI mode image processing circuit 1403 (step S12).

The image processing unit 15 then generates an ultrasonic image from the B-mode data generated by the B-mode processing unit 13 and the Doppler data generated by the Doppler processing unit 14. The image processing unit 15 also performs conversion (scan conversion) of a scanning line signal string for ultrasonic scanning into a scanning line signal string in a video format, and generates an ultrasonic image (a B-mode image, a Doppler image or an image obtained by superimposing them) as a display image (step S13).

As described above, this embodiment can provide an ultrasonic diagnostic apparatus which achieves an improvement in SNR and an increase in spatial resolution.

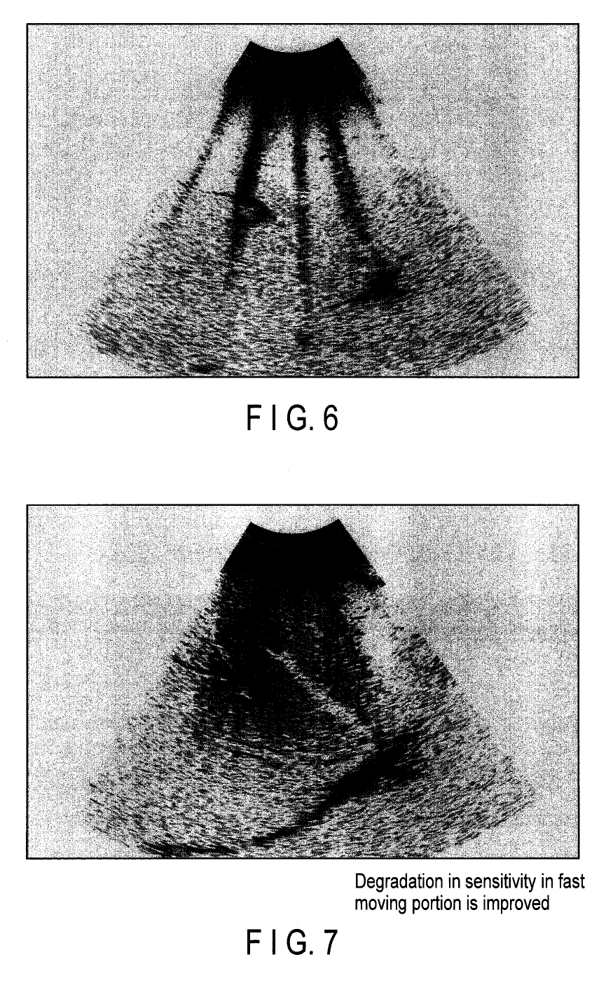

FIG. 6 is a view showing the ultrasonic image generated by a conventional ultrasonic diagnostic apparatus. FIG. 7 is a view showing the ultrasonic image generated by the ultrasonic diagnostic apparatus according to this embodiment. FIGS. 6 and 7 show ultrasonic images of the same in vivo tissue (diagnosis target region).

Referring to FIGS. 6 and 7, the black portions are those which cannot be imaged due to the motion of the diagnosis target region, and the white portions are those which can be imaged. As shown in FIGS. 6 and 7, it is obvious that the portions which cannot be imaged by the conventional ultrasonic diagnostic apparatus are imaged by the ultrasonic diagnostic apparatus according to this embodiment, and hence an improvement in SNR and an increase in spatial resolution are achieved.

Note that the ultrasonic diagnostic apparatus 100 according to this embodiment can be applied to a case in which it performs harmonic imaging based on the filter method as well as a case in which it performs harmonic imaging based on the polarity determination technique.

In addition, in the above embodiment, a plurality of reception signals concerning each scanning line are subjected to the determination processing performed by the threshold determination circuit 1301 and the delay correction processing performed by the delay circuit 1302. In contrast to this, for example, reception signals having phase differences equal to or less than a threshold (second threshold) need not be corrected because the influences of body motion and the like on phases can be neglected. Therefore, reception signals having phase differences equal to or less than the predetermined threshold (second threshold) may be excluded from the threshold determination circuit 1301 and the delay correction processing performed by the delay circuit 1302. This makes it possible to exclude reception signals which need not be delay-corrected and perform delay correction for only reception signals which need to be delay-corrected. This can improve the efficiency of arithmetic processing.

In addition, in the above embodiment, a plurality of reception signals concerning each scanning line in one frame (or one volume) are subjected to the threshold determination circuit 1301 and the delay correction processing performed by the delay circuit delay circuit 1302. However, the embodiment is not limited to this, and only a plurality of reception signals concerning a desired local region (e.g., at least one arbitrarily selected scanning line or region of interest (ROI)) in one frame (or one volume) may be subjected to the determination processing performed by the threshold determination circuit 1301 and the delay correction processing performed by the delay circuit 1302. In this case, the user may select a local region to be processed by manual operation or the apparatus may automatically select such a region based on information obtained by ultrasonic scanning, e.g., a region with a predetermined luminance or more or a region in which a large amount of motion is detected.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An ultrasonic diagnostic apparatus which generates an image based on reception signals acquired by ultrasonic transmission/reception of performing transmission of an ultrasonic beam and reception of reflected wave data on a plurality of scanning lines, the apparatus comprising:
a transmission/reception unit configured to acquire, for each scanning line, a plurality of reception signals corresponding to a plurality of ultrasonic beams by performing a plurality of times of ultrasonic transmission/reception on the same scanning line;
a phase difference detection unit configured to detect a phase difference between temporally adjacent reception signals of a plurality of reception signals on the each scanning line at each sampling point on the each scanning line;
a correction unit configured to perform correction processing including at least time delay correction for at least one of a plurality of reception signals for the each scanning line based on the phase difference at each sampling point on the each scanning line;
an addition unit configured to add, for the each scanning line, the plurality of reception signals including a reception signal subjected to the correction processing; and
an image generation unit configured to generate a first image from the reception signals added for the each scanning line.

2. The apparatus of claim 1, wherein the correction unit calculates, for the each reception signal, a delay amount from a predetermined time based on an accumulated phase difference between the plurality of reception signals and a period of a reception center frequency at each sampling point on the each scanning line, and executes the correction processing based on the each delay amount.

3. The apparatus of claim 1, wherein the correction unit calculates, for the each reception signal, a delay amount from a predetermined time based on any one of an average phase difference, a representative phase difference, and a median phase difference of the plurality of reception signals and a period of a reception center frequency, and executes the correction processing based on the each delay amount.

4. The apparatus of claim 1, wherein the image generation unit generates a second image based on a reception signal acquired by a Doppler mode, and
the apparatus further comprises a display unit configured to superimpose and display the first image and the second image.

5. The apparatus of claim 1, further comprising a determination unit configured to determine the reception signal having a phase difference not less than a first threshold at each sampling point on the each scanning line,
wherein the addition unit executes the addition by using reception signals other than a reception signal having a phase difference not less than the first threshold.

6. The apparatus of claim 1, further comprising a determination unit configured to determine the reception signal having a phase difference not less than a second threshold at each sampling point on the each scanning line,
wherein the correction unit executes the correction processing for a reception signal having a phase difference not less than the second threshold.

7. The apparatus of claim 1, wherein the correction unit executes the correction processing for at least one of a plurality of reception signals on a scanning line selected from the plurality of scanning lines.

8. The apparatus of claim 1, wherein the correction unit executes the correction processing for only the selected region based on the phase difference at a sampling point existing in a selected region.

9. The apparatus of claim 1, wherein the reception signal comprises an RF signal.

10. The apparatus of claim 1, wherein the reception signal comprises an IQ signal obtained by quadrature detection, and
the correction unit performs the correction processing including the time delay correction and phase delay correction.

11. A method of controlling an ultrasonic diagnostic apparatus which generates an image based on reception signals acquired by ultrasonic transmission/reception of performing transmission of an ultrasonic beam and reception of reflected wave data on a plurality of scanning lines, the method comprising:
acquiring, for each scanning line, a plurality of reception signals corresponding to a plurality of ultrasonic beams by performing a plurality of times of ultrasonic transmission/reception on the same scanning line;
detecting a phase difference between temporally adjacent reception signals of a plurality of reception signals on the each scanning line at each sampling point on the each scanning line;
performing correction processing including at least time delay correction for at least one of a plurality of reception signals for the each scanning line based on the phase difference at each sampling point on the each scanning line;
adding, for the each scanning line, the plurality of reception signals including a reception signal subjected to the correction processing; and
generating a first image from the reception signals added for the each scanning line.

12. The method of claim 11, wherein in the correcting, a delay amount is calculated, for the each reception signal, from a predetermined time based on an accumulated phase difference between the plurality of reception signals and a period of a reception center frequency at each sampling point on the each scanning line, and the correction processing is executed based on the each delay amount.

13. The method of claim 11, wherein in the correcting, a delay amount is calculated, for the each reception signal, from a predetermined time based on any one of an average phase difference, a representative phase difference, and a median phase difference of the plurality of reception signals and a period of a reception center frequency, and the correction processing is executed based on the each delay amount.

14. The method of claim 11, wherein in the generating, a second image is generated based on a reception signal acquired by a Doppler mode, and
the method further comprises superimposing and displaying the first image and the second image.

15. The method of claim 11, further comprising determining the reception signal having a phase difference not less than a first threshold at each sampling point on the each scanning line,
wherein in the adding, the addition is executed by using reception signals other than a reception signal having a phase difference not less than the first threshold.

16. The method of claim 11, further comprising determining the reception signal having a phase difference not less than a second threshold at each sampling point on the each scanning line,
wherein in the correcting, the correction processing is executed for a reception signal having a phase difference not less than the second threshold.

17. The method of claim 11, wherein in the correcting, the correction processing is executed for at least one of a plurality of reception signals on a scanning line selected from the plurality of scanning lines.

18. The method of claim 11, wherein in the correcting, the correction processing is executed for only the selected region based on the phase difference at a sampling point existing in a selected region.

19. The method of claim 11, wherein the reception signal comprises an RF signal.

20. The method of claim 11, wherein the reception signal comprises an IQ signal obtained by quadrature detection, and
in the correcting, the correction processing including the time delay correction and phase delay correction is performed.
